SEQUENCE LISTING (12 sequences)

SEQ ID NO: 1 - protein, 863 aa, synthetic construct
MKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRGENLYFQGGESFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTLEHHHHHH

SEQ ID NO: 2 - DNA, 1938 bp, synthetic construct
  source 1..1938: mol_type = other DNA
atgggtcgcggatccatgaaaactgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggggagaaaatctttattttcaaggtcatcatcatcatcatcatcatggagaaagctttagagttcagccaacagagagcatcgtgcgcttcccaaacatcaccaacctgtgcccttttggagaagtgtttaatgccaccagatttgctagcgtgtacgcctggaacagaaaaagaatcagcaactgtgtggctgactactctgtgctgtacaacagcgcctccttctccaccttcaagtgctacggtgtgtccccaaccaagctgaatgacctgtgcttcaccaatgtttatgctgacagctttgtcatcagaggagatgaagtgcggcagattgctcctggccagacaggcaagattgctgattacaactacaagctgcctgatgacttcacaggctgtgtcatcgcctggaacagcaacaacctggacagcaaggtgggcggcaactacaactacctgtacagacttttcaggaagagcaatctgaagccatttgaaagagacatcagcacagagatctaccaggctggcagcacaccctgcaatggagtggaaggcttcaactgctactttcctctgcagagctacggcttccagcccaccaacggcgtgggctaccagccttacagagtggtggtgctgtcctttgagctgctgcacgcccctgccacagtgtgtggccccaagaagtctaccaacctggtgaagaacaaatgtgtgaacttcaatttcaatggcctgacaggcacaggactcgagtga

SEQ ID NO: 3 - protein, 419 aa, synthetic construct
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 4 - DNA, 1260 bp, synthetic construct
  source 1..1260: mol_type = other DNA
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 5 - protein, 123 aa, synthetic construct
QVQLVESGGGSVQAGGSLRLSCIASGTFPFRFNAMAWFRQAPGNQRELVAGIFTTYATNYAESVKGRFTISRDNEKNTIYLQMNSLKPEDTGVYYCNAAYVSTSIWRPLYEEWGQGTQVTVSS

SEQ ID NO: 6 - protein, 112 aa, synthetic construct
GLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLE

SEQ ID NO: 7 - protein, 127 aa, synthetic construct
QVQLVESGGALMQAGGSLRLSCAVSGIRLGYYTIAWFRQAPGKEREGISCISRSEGSTYYADSVKGRFTISTENAENTVYLQMNSLKPDDTAVYYCAVDFVPASHCAVANTKGYDYWGQGTQVTVSS

SEQ ID NO: 8 - protein, 100 aa, synthetic construct
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGK

SEQ ID NO: 9 - protein, 125 aa, synthetic construct
QVQLVESGGGSVQAGGSLRLSCAASGSISSITYLGWFRQAPGKEREGVAALITVQAHTYYADSVKGRFTISRDNAKNTVYLQMNSLKPEDTALYYCAAAAFGYAWPLRQDDYWYWGQGTQVTVSS

SEQ ID NO: 10 - protein, 73 aa, synthetic construct
YNSASFSTFKCYGVSPTKLNDLCFTNVYADSFVIRGDEVRQIAPGQTGKIADYNYKLPDDFTGCVIAWNSNNL

SEQ ID NO: 11 - protein, 125 aa, synthetic construct
QVQLVESGGGSVQAGGSLRLSCIGSARTFHSYVMAWFRQAPGKEREFVAAISWSSTPTYYGESVKGRFTISRDNAKNTVYLQMNSLKPEDTAVYFCAADRADSYYHTRPTDYEFWGQGTQVTVSS

SEQ ID NO: 12 - protein, 33 aa, synthetic construct
QAGSTPCNGVEGFNCYFPLQSYGFQPTNGVGYQ

(19) United States
(12) Patent Application Publication
(10) Pub. No.: US 2025/0155438 A1
(43) Pub. Date: May 15, 2025

(54) DEVICE AND METHOD FOR PATHOGEN DETECTION

(71) Applicant: King Abdulaziz University, Jeddah (SA)

(72) Inventors: Jamal Sabir M. Sabir, Jeddah (SA); Ahmed Atef, Jeddah (SA); Abd AB El-Razik, Jeddah (SA); Sherif Edris, Jeddah (SA); Alawiyah M. ALhibshi, Jeddah (SA); Nahid H. Hajarah, Jeddah (SA); Ahmed Bahieldin, Jeddah (SA); Masjid Rasool Kamli, Jeddah (SA); Irfan A. Rather, Jeddah (SA)

(21) Appl. No.: 19/018,238

(22) Filed: Jan. 13, 2025

Related U.S. Application Data
(63) Continuation-in-part of application No. 18/333,596, filed on Jun. 13, 2023.

(51) Int. Cl. G01N 33/569; C07K 16/10; G01N 33/543

(52) U.S. Cl. CPC G01N 33/56983; C07K 16/1003; G01N 33/54388; C07K 2317/569; G01N 2333/165; G01N 2469/10

(57) ABSTRACT

Systems and methods are disclosed herein for pathogen detection employing a lateral flow assay (LFA) device or ELISA assay, e.g. for detecting SARS-COV-2, the virus that causes COVID-19, in a sample. The LFA device includes a nitrocellulose membrane mounted on a solid support, a sample pad for receiving a sample, a conjugate pad containing gold nanoparticles conjugated to heavy chain antibodies (HcAbs), and an absorbent pad at the end of the device.

### CROSS-REFERENCE TO RELATED APPLICATIONS

This application is a continuation-in-part of U.S. patent application Ser. No. 18/333,596 filed Jun. 13, 2023, the contents of which are incorporated herein by reference.

### SEQUENCE LISTING

This document incorporates by reference an electronic sequence listing text file, which was electronically submitted along with this document. The xml file is named 2025-01-13_16040005C1_seqlisting.xml, is 16628 bytes, and was created on Jan. 13, 2025.

### FIELD OF THE INVENTION

The present invention relates to the detection and characterization of pathogens, viruses, and other biological materials.

### BACKGROUND

Pathogens constitute a critical problem for human, animal and plant health. Pathogens may cause infections that result in a variety of human illnesses and can lead to a large number of deaths. Such pathogens may include viruses, bacteria, prions, fungi, molds, eukaryotic microbes and parasites of many types. Moreover, pathogens infect agriculturally important plant and animal species, resulting in economic hardship. Detection and identification of pathogens in relevant materials (e.g., water, air, blood, tissues, organs, etc.) is essential to minimize the transfer and spread of infections. Furthermore, quick identification may aid in devising effective treatment strategies.

One exemplary class of pathogens is viruses. Viral infections extol a great morbidity and mortality among the human population. Many of these infections result from undetected viruses in water, food and air and are promulgated by an ever-increasing interconnection of societies. Detection and identification in medically important materials such as blood, blood derivatives, tissues and organs is critical to minimize the potential for transfer and spread within hospitals and clinics and to the staff of these centers.

Several popular methods for the detection and identification of viruses and pathogens exist. These generally fall into three categories: A) Infectivity and infectivity reduction assays; B) Serology assays employing antibody detection to determine whether an individual has been exposed; and C) Direct virology assays in which antibodies are used to detect the presence of an antigen in the sample or nucleic acid-based assays in which elements of the viral genome are detected. Infectivity-based assays are seldom used in diagnostics yet, both cell culture and animal-based amplification of virus in a sample may be necessary for many of the current diagnostic procedures. The use of animals in infectivity assays is costly, time consuming and subject to ethical dispute. Serodiagnosis still exists in many hospitals principally because there are no good alternatives for some infections. Serology is largely performed to determine antibody levels and to estimate the probability for infection. Antibody based tests are popular but are usually limited to a battery of individual tests in a macroscopic format (e.g., Enzyme Linked ImmunoSorbant Assay, or ELISA). Standard microbiological approaches to detect anthrax and other bacterial pathogens involve growth of the agent on nutrient agar and visual identification after various staining procedures. Carbon source utilization testing in various media identifies and differentiates among closely related isolates. Viral pathogens are usually identified after their administration, infection and amplification in animals, particularly embryonated eggs, mice or cell culture. This is the basic microbial identification scheme practiced today. While precise in their verification of pathogen identity, these procedures are very slow.

Multiple advancements have been seen in similar regards. For instance, a CA patent 2,395,318C discusses a method and apparatus for detection of microscopic pathogens. Such apparatus includes: a substrate with a detection region on a surface thereof, the detection region having microstructures including grooves formed therein that will align liquid crystal material in contact therewith, the width and depth of the grooves being in the range of 10 μm or less; a blocking layer on the surface of the detection region of the substrate that does not disrupt the alignment of liquid crystal material in contact therewith, the blocking layer blocking nonspecific adsorption of pathogens to the surface; and a binding agent on the surface of the detection region of the substrate, the binding agent specifically binding the selected pathogen,

US patent application 2002/0172943A1 (issued as U.S. Pat. No. 6,897,015) relates to a device and method of use for detection and characterization of pathogens and biological materials. The method and apparatus includes providing a substrate with a surface and forming domains of deposited materials thereon. The deposited material can be placed on the surface and bound directly and non-specifically to the surface, or it may be specifically or non-specifically bound to the surface. The deposited material has an affinity for a specific target material. The domains thus created are termed affinity domains or deposition domains. Multiple affinity domains of deposited materials can be deposited on a single surface, creating a plurality of specific binding affinity domains for a plurality of target materials. Target materials may include, for example, pathogens or pathogenic markers such as viruses, bacteria, bacterial spores, parasites, prions, fungi, mold or pollen spores. The device thus created is incubated with a test solution, gas or other supporting environment suspected of containing one or more of the target materials. Specific binding interactions between the target materials and a particular affinity domain occurs and is detected by various methods.

U.S. Pat. No. 11,179,061 discusses methods and devices for detecting viruses and bacterial pathogens. The embodiments disclose a method including making electrodes using an electrically conductive material for impedimetric detection of analytical targets in an electrochemical sensing platform device, collecting patient samples in a solution compartment of the electrochemical sensing platform device, binding primers and aptamers to electrodes for functionalizing electrodes for detecting and binding targeted viruses and bacterial pathogens, incubating oral fluid samples in the solution compartment using a heater, measuring electrode impedance changes, recording measured electrode impedance changes in a memory device, and integrating wireless technologies into the electrochemical sensing platform device configured for transmitting recorded measured data,

Novel devices and methods for pathogen detection are needed.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specifications, claims, drawings, and abstract as a whole.

Provided herein is a lateral flow assay (LFA) device for detecting specific molecules in a sample. The LFA also includes a test region on a porous membrane where the capture heavy chain antibodies (HcAbs), which are specifically designed to bind to the target antigen, are immobilized. A control region on the membrane, e.g. containing rabbit anti-camel IgG, is also provided to ensure the accuracy of the test. The disclosed LFA device reduces the complexity of pathogen detection.

One aspect of the disclosure provides an LFA device for pathogen detection, comprising a sample pad at a first end of the device for receiving a sample; a conjugate pad containing gold nanoparticles conjugated to heavy chain antibodies (HcAbs), wherein the conjugate pad partially overlaps the sample pad; a porous membrane containing a test region and a control region; and an absorbent pad at a second end of the device, wherein the porous membrane partially overlaps the conjugate pad and the absorbent pad and wherein each of the sample pad, conjugate pad, porous membrane, and absorbent pad are at least partially mounted on a solid support. The HcAbs may be obtained by immunizing a camelid with a target antigen, and affinity purifying HcAbs from camelid serum from the immunized camelid.

In some embodiments, the HcAbs are obtained from an immunized camel. In some embodiments, the control region comprises immobilized anti-camel IgG. In some embodiments, the test region comprises immobilized capture HcAbs configured to bind a target antigen. In some embodiments, the target antigen is viral spike glycoprotein or nucleocapsid protein. In some embodiments, the target antigen is a SARS-Cov-2 viral protein. In some embodiments, the porous membrane is a nitrocellulose membrane. In some embodiments, the HcAbs comprise a paratope having a sequence at least 90% identical to a sequence selected from the group consisting of SEQ ID NO: 6, SEQ ID NO: 8, SEQ ID NO: 10, and SEQ ID NO: 12. In some embodiments, the HcAbs comprise three complementarity determining regions (CDRs) having a sequence at least 90% identical to CDR sequences as set forth in SEQ ID NO: 5, SEQ ID NO: 7, SEQ ID NO: 9, or SEQ ID NO: 11.

Another aspect of the disclosure provides a method of detecting a pathogen in a biological sample, comprising contacting the biological sample with an LFA device as described herein.

Another aspect of the disclosure provides a heavy chain antibody having a sequence as described herein.

Another aspect of the disclosure provides an ELISA assay utilizing the heavy chain antibodies as described herein. In some embodiments, the disclosure provides a microplate with wells pre-coated with immobilized heavy chain antibodies. In some embodiments, the heavy chain antibody is biotinylated and is bound to streptavidin coating the well.

Another aspect of the disclosure provides a method of detecting a pathogen in a biological sample, comprising contacting the biological sample with a heavy chain antibody as described herein.

Additional features and advantages of the present invention will be set forth in the description of disclosure that follows, and in part, will be apparent from the description of may be learned by practice of the disclosure. The disclosure will be realized and attained by the compositions and methods particularly pointed out in the written description and claims hereof.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention and explain various principles and advantages of those embodiments.

FIGS. 1A-B is a schematic representation of a lateral flow strip immobilized with camel anti-Spike and N-capsid and Rabbit anti-camel IgGn. The strip is also immobilized with a viral protein coated with a gold particle as per preferred embodiments of the invention. A representation of serum taken from (A) a patient and (B) a healthy individual is provided.

FIG. 2 is a schematic of an exemplary configuration of a lateral flow assay test strip.

FIGS. 3A-B is a photograph of the reaction on the LFA strips for (A) a patient and (B) a healthy individual as per preferred embodiments of the invention.

FIG. 4 is a schematic representation of exemplary steps for a method as per preferred embodiments of the invention.

FIGS. 5A-D is a validation of nanobody pairs in ELISA assays for detection of SARS-CoV-2 NCAP and RBD proteins. FIGS. 5A and 5C display the SDS-PAGE images of RBD and NCAP sandwich assays, respectively, illustrating the different proteins used in this experiment. The images highlight the purity and integrity of the proteins, confirming their effective use in the antigen capture and detection process. (5B) The anti-RBD-1/RBD-2 nanobody pair exhibits higher signal intensity than the previously published anti-RBD monoclonal antibody pair SB68/SB45. (5D) The anti-NCAP-1/NCAP-2 nanobody pair exhibits slightly lower signal intensity than the previously published anti-NCAP monoclonal antibody pair 2H4/2G3. For all assays, antigen capture was carried out using biotinylated nanobodies immobilized on streptavidin-coated plates. Detection of bound nanobodies was achieved with a commercial rat-derived anti-HA-HRP antibody (1:5000; Roche) or anti-mouse IgG HRP-linked antibody (1:5000; Abcam). Background signals were minimal in wells containing BSA, as indicated by the control experiments (5B and 5D).

FIG. 6 is an example ELISA microplate.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

Detailed descriptions of the preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The preferred embodiments of the present disclosure are directed toward a simple, portable, and cost-effective tool for detecting target molecules in a sample. In particular, the disclosure provides a lateral flow assay (LFA) useful for detecting pathogens. LFAs are a rapid testing platform for the detection and quantification of analytes in complex mixtures, where the sample is placed on a test device and the results are displayed within 1-40 minutes, e.g. 5-30 minutes or 10-25 minutes. The detected analytes may include antigens, antibodies, and products of gene amplification.

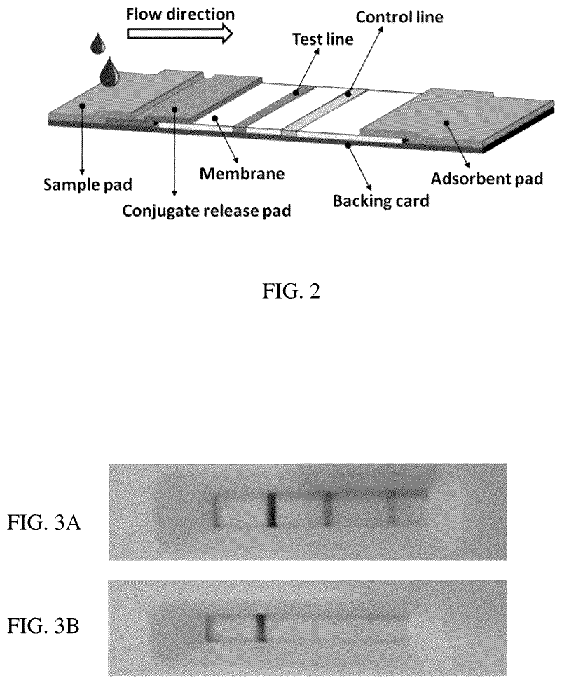

With reference to FIG. 2, the general configuration of an LFA test strip includes overlapping membranes that are mounted on a backing card for better stability and handling. The overlapping membranes include a sample pad at a first end of the device for receiving a sample; a conjugate pad, wherein the conjugate pad partially overlaps the sample pad; a nitrocellulose membrane containing a test region and a control region; and an absorbent pad at a second end of the device, wherein the nitrocellulose membrane partially overlaps the conjugate pad and the absorbent pad and wherein each of the sample pad, conjugate pad, nitrocellulose membrane, and absorbent pad are at least partially mounted on a solid support, e.g. a paper, plastic, or metal backing.

In use, a liquid sample containing the analyte of interest moves without the assistance of external forces (capillary action) through various zones of polymeric strips, on which molecules that can interact with the analyte are attached. The sample is applied at one end of the strip, on the adsorbent sample pad, e.g. a glass fiber pad, which may be impregnated with buffer salts, surfactants, proteins, or other liquids to control the flow rate of the sample and make the sample suitable for interaction with the detection system. In some embodiments, the sample pad is impregnated with bovine serum albumin (BSA). The pores of the sample pad can act as a filter to remove redundant materials such as red blood cells. In some embodiments, the sample pad contains as aggregating agent, for example, a sugar such as mannitol, sorbitol or inositol, one or more red blood cell-binding antibodies, lectins, or the like. The sample pad ensures that the analyte present in the sample will be capable of binding to the capture reagents on the conjugate pad and on the membrane.

The treated sample migrates through the conjugate pad, e.g. a glass fiber pad, which contains antibodies that are specific to the target analyte and are conjugated to coloured or fluorescent particles. The conjugate pad may contain be impregnated with a conjugate buffer, e.g. containing carbohydrates such as sucrose which serve as a preservative and a resolubilization agent. When the conjugate particles are dried in the presence of sugar, the sugar molecules form a layer around them stabilizing their biological structures. When the sample enters the conjugate pad, the sugar molecules rapidly dissolve carrying the particles into the fluid stream. In preferred embodiments, the conjugate pad contains gold nanoparticles conjugated to heavy chain antibodies (HcAbs) which bind the analyte of interest. The gold nanoparticles may have a particle size of 20-50 nm. Alternative nanoparticle labels include, but are not limited to, silver nanoparticles, quantum dots, magnetic nanoparticles, etc.

A heavy-chain antibody (also known as a nanobody®, single domain antibody, or V_{H}H antibody) is an antibody which contains only two heavy chains and lacks the two light chains usually found in antibodies. HcAbs can bind antigens despite having only V_{H} domains. The only mammals with heavy-chain (IgG-like) antibodies are camelids such as dromedaries, camels, llamas and alpacas. They make up around 80% of the circulating antibodies in the serum of camels. Heavy-chain camelid antibodies can be just as specific as traditional antibodies and in some cases they are more robust. Further, they are easily isolated using the same phage panning procedure used for traditional antibodies, allowing them to be cultured ex vivo in large concentrations. In some embodiments, HcAbs are prepared using a prokaryotic expression system as is known in the art. In some embodiments, the HcAbs are obtained by immunizing a camelid with a target antigen, and affinity purifying HcAbs from camelid serum from the immunized camelid.

HcAbs have smaller antigen-binding sites (Fab domains) and more flexible CDR3 regions compared to traditional antibodies, which allows them to bind to epitopes that may not be accessible to traditional antibodies. In addition, HcAbs have higher rates of mutation in their CDR3 region and non-canonical cysteine pairs in their CDRs and frameworks, which increases their stability and diversity. HcAbs are also able to withstand extreme conditions without losing their ability to bind to designated antigens. For example, anti-caffeine HcAbs were able to accurately determine caffeine levels in beverages heated to 70° C., and HcAbs detecting a red azo dye was active in binding assays at 90° C. The thermal stability of HcAbs also allows for a prolonged shelf life.

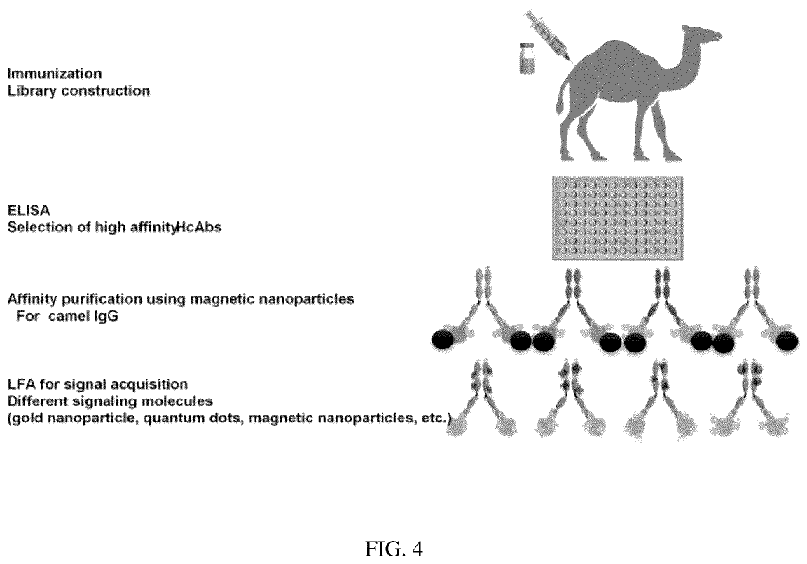

With reference to FIG. 4, the HcAbs described herein may be obtained by immunizing a camelid with an antigen, e.g. a viral protein, followed by selection and purification of highly specific antibodies. In some embodiments, the target antigen is viral spike glycoprotein or nucleocapsid protein. In some embodiments, the target antigen is a coronavirus viral protein, such as a viral protein from SARS-CoV, SARS-CoV-2, MERS-CoV, HCoV-OC43, HCoV-HKU1, HCoV-229E, or HCoV-NL63. These viral proteins play crucial roles in the replication, assembly, and immune response of coronaviruses. Specific antigens from human coronaviruses (HCoVs) such as HCov-OC43, HCoV-HKU1, HCoV-229E, and HCoV-NL63 can also be targeted. These are human respiratory coronaviruses and known to cause mild to moderate respiratory illnesses. Examples of antigens that can be utilized for these HCoV include: HCoV-OC43 spike glycoprotein, HCoV-HKU1 spike glycoprotein, HCoV-229E spike glycoprotein, HCoV-NL63 spike glycoprotein, HCoV-OC43 nucleocapsid protein, HCoV-HKU1 nucleocapsid protein, HCoV-229E nucleocapsid protein, HCoV-NL63 nucleocapsid protein, HCoV-OC43 membrane protein, HCoV-HKU1 membrane protein, HCoV-229E membrane protein, and HCoV-NL63 membrane protein. Utilizing them as antigens allows to target specific components of the virus and generate antibodies that can bind to and neutralize the virus or aid in diagnostic assays.

In the Example, the following antigen sequences for SARS-COV-2 Spike S1 domain protein and nucleocapsid protein were used:

MBP_S-RBD
(SEQ ID NO: 1)
MKTEEGKLVIWINGDKGYNGLAEVGKKFEKDTGIKVTVEHPDKLEEKFP
QVAATGDGPDIIFWAHDRFGGYAQSGLLAEITPDKAFQDKLYPFTWDAV
RYNGKLIAYPIAVEALSLIYNKDLLPNPPKTWEEIPALDKELKAKGKSA
LMFNLQEPYFTWPLIAADGGYAFKYENGKYDIKDVGVDNAGAKAGLTFL
VDLIKNKHMNADTDYSIAEAAFNKGETAMTINGPWAWSNIDTSKVNYGV
TVLPTFKGQPSKPFVGVLSAGINAASPNKELAKEFLENYLLTDEGLEAV
NKDKPLGAVALKSYEEELAKDPRIAATMENAQKGEIMPNIPQMSAFWYA
VRTAVINAASGRQTVDEALKDAQTNSSSNNNNNNNNNNLGIEGRGENLY
FQGGESFIEDLLFNKVTLADAGFIKQYGDCLGDIAARDLICAQKFNGLT
VLPPLLTDEMIAQYTSALLAGTITSGWTFGAGAALQIPFAMQMAYRENG
IGVTQNVLYENQKLIANQFNSAIGKIQDSLSSTASALGKLQDVVNQNAQ
ALNTLVKQLSSNFGAISSVLNDILSRLDKVEAEVQIDRLITGRLQSLQT
YVTQQLIRAAEIRASANLAATKMSECVLGQSKRVDFCGKGYHLMSFPQS
APHGVVFLHVTYVPAQEKNFTTAPAICHDGKAHFPREGVFVSNGTHWFV
TQRNFYEPQIITTDNTFVSGNCDVVIGIVNNTVYDPLQPELDSFKEELD
KYFKNHTSPDVDLGDISGINASVVNIQKEIDRLNEVAKNLNESLIDLQE
LGKYEQYIKWPWYIWLGFIAGLIAIVMVTIMLCCMTSCCSCLKGCCSCG
SCCKFDEDDSEPVLKGVKLHYTLEHHHHHH

Residues 1-388 are the maltose binding protein domain, residues 389-397 are the TEV protease cleavage site, and residues 398-863 are the spike receptor binding domain. The spike receptor binding domain separated from the expressed protein using TEV protease was used for immunization.

MBP_S-RBD
(SEQ ID NO: 2)
ATGGGTCGCGGATCCATGAAAACTGAAGAAGGTAAACTGGTAATCTGGA
TTAACGGCGATAAAGGCTATAACGGTCTCGCTGAAGTCGGTAAGAAATT
CGAGAAAGATACCGGAATTAAAGTCACCGTTGAGCATCCGGATAAACTG
GAAGAGAAATTCCCACAGGTTGCGGCAACTGGCGATGGCCCTGACATTA
TCTTCTGGGCACACGACCGCTTTGGTGGCTACGCTCAATCTGGCCTGTT
GGCTGAAATCACCCCGGACAAAGCGTTCCAGGACAAGCTGTATCCGTTT
ACCTGGGATGCCGTACGTTACAACGGCAAGCTGATTGCTTACCCGATCG
CTGTTGAAGCGTTATCGCTGATTTATAACAAAGATCTGCTGCCGAACCC
GCCAAAAACCTGGGAAGAGATCCCGGCGCTGGATAAAGAACTGAAAGCG
AAAGGTAAGAGCGCGCTGATGTTCAACCTGCAAGAACCGTACTTCACCT
GGCCGCTGATTGCTGCTGACGGGGGTTATGCGTTCAAGTATGAAAACGG
CAAGTACGACATTAAAGACGTGGGCGTGGATAACGCTGGCGCGAAAGCG
GGTCTGACCTTCCTGGTTGACCTGATTAAAAACAAACACATGAATGCAG
ACACCGATTACTCCATCGCAGAAGCTGCCTTTAATAAAGGCGAAACAGC
GATGACCATCAACGGCCCGTGGGCATGGTCCAACATCGACACCAGCAAA
GTGAATTATGGTGTAACGGTACTGCCGACCTTCAAGGGTCAACCATCCA
AACCGTTCGTTGGCGTGCTGAGCGCAGGTATTAACGCCGCCAGTCCGAA
CAAAGAGCTGGCAAAAGAGTTCCTCGAAAACTATCTGCTGACTGATGAA
GGTCTGGAAGCGGTTAATAAAGACAAACCGCTGGGTGCCGTAGCGCTGA
AGTCTTACGAGGAAGAGTTGGCGAAAGATCCACGTATTGCCGCCACCAT
GGAAAACGCCCAGAAAGGTGAAATCATGCCGAACATCCCGCAGATGTCC
GCTTTCTGGTATGCCGTGCGTACTGCGGTGATCAACGCCGCCAGCGGTC
GTCAGACTGTCGATGAAGCCCTGAAAGACGCGCAGACTAATTCGAGCTC
GAACAACAACAACAATAACAATAACAACAACCTCGGGATCGAGGGAAGG
GGAGAAAATCTTTATTTTCAAGGTCATCATCATCATCATCATCATGGAG
AAAGCTTTAGAGTTCAGCCAACAGAGAGCATCGTGCGCTTCCCAAACAT
CACCAACCTGTGCCCTTTTGGAGAAGTGTTTAATGCCACCAGATTTGCT
AGCGTGTACGCCTGGAACAGAAAAAGAATCAGCAACTGTGTGGCTGACT
ACTCTGTGCTGTACAACAGCGCCTCCTTCTCCACCTTCAAGTGCTACGG
TGTGTCCCCAACCAAGCTGAATGACCTGTGCTTCACCAATGTTTATGCT
GACAGCTTTGTCATCAGAGGAGATGAAGTGCGGCAGATTGCTCCTGGCC
AGACAGGCAAGATTGCTGATTACAACTACAAGCTGCCTGATGACTTCAC
AGGCTGTGTCATCGCCTGGAACAGCAACAACCTGGACAGCAAGGTGGGC
GGCAACTACAACTACCTGTACAGACTTTTCAGGAAGAGCAATCTGAAGC
CATTTGAAAGAGACATCAGCACAGAGATCTACCAGGCTGGCAGCACACC
CTGCAATGGAGTGGAAGGCTTCAACTGCTACTTTCCTCTGCAGAGCTAC
GGCTTCCAGCCCACCAACGGCGTGGGCTACCAGCCTTACAGAGTGGTGG
TGCTGTCCTTTGAGCTGCTGCACGCCCCTGCCACAGTGTGTGGCCCCAA
GAAGTCTACCAACCTGGTGAAGAACAAATGTGTGAACTTCAATTTCAAT
GGCCTGACAGGCACAGGACTCGAGTGA
N-capsid
(SEQ ID NO: 3)
MSDNGPQNQRNAPRITFGGPSDSTGSNQNGERSGARSKQRRPQGLPNNT
ASWFTALTQHGKEDLKFPRGQGVPINTNSSPDDQIGYYRRATRRIRGGD
GKMKDLSPRWYFYYLGTGPEAGLPYGANKDGIIWVATEGALNTPKDHIG
TRNPANNAAIVLQLPQGTTLPKGFYAEGSRGGSQASSRSSSRSRNSSRN
STPGSSRGTSPARMAGNGGDAALALLLLDRLNQLESKMSGKGQQQQGQT
VTKKSAAEASKKPRQKRTATKAYNVTQAFGRRGPEQTQGNFGDQELIRQ
GTDYKHWPQIAQFAPSASAFFGMSRIGMEVTPSGTWLTYTGAIKLDDKD
PNFKDQVILLNKHIDAYKTFPPTEPKKDKKKKADETQALPQRQKKQQTV
TLLPAADLDDFSKQLQQSMSSADSTQA
N-capsid
(SEQ ID NO: 4)
ATGTCTGATAATGGACCCCAAAATCAGCGAAATGCACCCCGCATTACGT
TTGGTGGACCCTCAGATTCAACTGGCAGTAACCAGAATGGAGAACGCAG
TGGGGCGCGATCAAAACAACGTCGGCCCCAAGGTTTACCCAATAATACT
GCGTCTTGGTTCACCGCTCTCACTCAACATGGCAAGGAAGACCTTAAAT
TCCCTCGAGGACAAGGCGTTCCAATTAACACCAATAGCAGTCCAGATGA
CCAAATTGGCTACTACCGAAGAGCTACCAGACGAATTCGTGGTGGTGAC
GGTAAAATGAAAGATCTCAGTCCAAGATGGTATTTCTACTACCTAGGAA
CTGGGCCAGAAGCTGGACTTCCCTATGGTGCTAACAAAGACGGCATCAT
ATGGGTTGCAACTGAGGGAGCCTTGAATACACCAAAAGATCACATTGGC
ACCCGCAATCCTGCTAACAATGCTGCAATCGTGCTACAACTTCCTCAAG
GAACAACATTGCCAAAAGGCTTCTACGCAGAAGGGAGCAGAGGCGGCAG
TCAAGCCTCTTCTCGTTCCTCATCACGTAGTCGCAACAGTTCAAGAAAT
TCAACTCCAGGCAGCAGTAGGGGAACTTCTCCTGCTAGAATGGCTGGCA
ATGGCGGTGATGCTGCTCTTGCTTTGCTGCTGCTTGACAGATTGAACCA
GCTTGAGAGCAAAATGTCTGGTAAAGGCCAACAACAACAAGGCCAAACT
GTCACTAAGAAATCTGCTGCTGAGGCTTCTAAGAAGCCTCGGCAAAAAC
GTACTGCCACTAAAGCATACAATGTAACACAAGCTTTCGGCAGACGTGG
TCCAGAACAAACCCAAGGAAATTTTGGGGACCAGGAACTAATCAGACAA
GGAACTGATTACAAACATTGGCCGCAAATTGCACAATTTGCCCCCAGCG
CTTCAGCGTTCTTCGGAATGTCGCGCATTGGCATGGAAGTCACACCTTC
GGGAACGTGGTTGACCTACACAGGTGCCATCAAATTGGATGACAAAGAT
CCAAATTTCAAAGATCAAGTCATTTTGCTGAATAAGCATATTGACGCAT
ACAAAACATTCCCACCAACAGAGCCTAAAAAGGACAAAAAGAAGAAGGC
TGATGAAACTCAAGCCTTACCGCAGAGACAGAAGAAACAGCAAACTGTG
ACTCTTCTTCCTGCTGCAGATTTGGATGATTTCTCCAAACAATTGCAAC
AATCCATGAGCAGTGCTGACTCAACTCAGGCCTAA

In the LFA device, the sample, together with the conjugated HcAbs bound to the target analyte, migrates along the strip into a porous membrane. The porous membrane may be formed from nitrocellulose, cellulose acetate, or a polymer including nylon, silicone, or the like. In some embodiments, the membrane has a pore size of 0.05 to 30 μm. The membrane contains specific biological components (e.g. antibodies or antigens) immobilized in lines. Their role is to react with the analyte bound to the conjugated antibody. Recognition of the sample analyte results in an appropriate response on the test line, while a response on the control line indicates the proper liquid flow through the strip. For example, if the LFA device contains HcAbs which recognize SARS-CoV-2 viral proteins and such viral proteins are present in the sample, they will bind to the HcAbs in the test region, causing a visible color change, e.g. from red to purple, within about 10-25 minutes. The sample then flows further to the control line which contains anti-camel IgG, where a similar color change indicates that the device is functioning properly. Thus, the disclosed method allows for the rapid and accurate detection of SARS-COV-2 in a sample.

In some embodiments, the HcAbs comprise a variable heavy chain domain (VHH) wherein the VHH comprises a frame region (FR) and a complementary determination region (CDR), where the CDR includes CDR1, CDR2 and CDR3, as well as FR1, FR2, FR3, FR4 separated by the CDR 1-3.

In some embodiments, the HcAbs comprise the following sequences or sequences having at least 80% identity to the following sequences, e.g. at least 80%, 85%, 90%, 95%, 96%, 97%, 98%, 99% identity. It is noted that the first sequence for each is the sequence of the variable region of the heavy chain that defines the antibody's specificity. The paratope is the region of the antibody that interacts directly with the antigen (e.g. the SARS-COV-2 nucleocapsid protein or receptor binding domain) and delineate the molecular interfaces that engage directly with the antigenic epitopes. The 3 CDRs of each HcAb are displayed in emboldened typography and underlined below.

```
N Capsid nanobodies
>NCAP-1
(SEQ ID NO: 5)
QVQLVESGGGSVQAGGSLRLSCIASGTFPFRFNAMAWFRQAPGNQRELVA
                                                      CDR1
GIFTTYATNYAESVKGRFTISRDNEKNTIYLQMNSLKPEDTGVYYCNAAY
  CDR2
VSTSIWRPLYEEWGQGTQVTVSS
CDR3
Paratope
(SEQ ID NO: 6)
GLPYGANKDGIIWVATEGALNTPKDHIGTRNPANNAAIVLQLPQGTTLPK
GFYAEGSRGGSQASSRSSSRSRNSSRNSTPGSSRGTSPARMAGNGGDAAL
ALLLLDRLNQLE
>NCAP-2
(SEQ ID NO: 7)
QVQLVESGGALMQAGGSLRLSCAVSGIRLGYYTIAWFRQAPGKEREGIS
                                                    CDR1
CISRSEGSTYYADSVKGRFTISTENAENTVYLQMNSLKPDDTAVYYC
  CDR2
AVDFVPASHCAVANTKGYDYWGQGTQVTVSS
        CDR3
Paratope
(SEQ ID NO: 8)
MSDNGPQNQRNAPRITFGGPSDSTGSNQNGERSGARSKQRRPQGLPNNTA
SWFTALTQHGKEDLKFPRGQGVPINTNSSPDDQIGYYRRATRRIRGGDGK
RBD nanobodies
>RBD-1
(SEQ ID NO: 9)
QVQLVESGGGSVQAGGSLRLSCAASGSISSITYLGWFRQAPGKEREGVA
                                                    CDR1
ALITVQAHTYYADSVKGRFTISRDNAKNTVYLQMNSLKPEDTALYYCAA
        CDR2
AAFGYAWPLRQDDYWYWGQGTQVTVSS
      CDR3
Paratope
(SEQ ID NO: 10)
YNSASFSTFKCYGVSPTKLNDLCFTNVYADSFVIRGDEVRQIAPGQTGKI
ADYNYKLPDDFTGCVIAWNSNNL
>RBD-2
(SEQ ID NO: 11)
QVQLVESGGGSVQAGGSLRLSCIGSARTFHSYVMAWFRQAPGKEREFVA
                                                      CDR1
AISWSSTPTYYGESVKGRFTISRDNAKNTVYLQMNSLKPEDTAVYFCAA
      CDR2
DRADSYYHTRPTDYEFWGQGTQVTVSS
        CDR3
Paratope
(SEQ ID NO: 12)
QAGSTPCNGVEGENCYFPLQSYGFQPTNGVGYQ
```

The HcAbs described herein may have varying degrees of homology to the specified sequences, e.g. with identity thresholds ranging from 80% to 99%. In some embodiments, the HcAbs comprise CDRs having at least at least 80%, 85%, 90%, 95%, 96%, 97%, 98%, or 99% identity with the CDRs set forth in SEQ ID Nos 5, 7, 9, and 11, e.g. by substituting 1, 2, 3, 4, or more amino acids. Various changes may be made in the amino acid sequences disclosed herein, or corresponding DNA sequences which encode said amino acid sequences, without appreciable loss of their biological activity.

Amino acid substitutions are generally based on the relative similarity of the amino acid side-chain substituents, for example, their hydrophobicity, hydrophilicity, charge, size, and the like. Exemplary conservative substitutions which take various of the foregoing characteristics into consideration are well known to those of skill in the art and include: arginine and lysine; glutamate and aspartate; serine and threonine; glutamine and asparagine; and valine, leucine and isoleucine. For example, a conservative mutation may involve substituting threonine at position 58 of SEQ ID NO: 5 with serine or substituting alanine at position 61 of SEQ ID NO: 5 with aspartic acid to preserve the hydrophilic character of the loop. As another example, a conservative mutation may involve substituting glutamic acid at position 55 of SEQ ID NO: 7 with glutamine to maintain antigen recognition. Further, conservative mutations may include substituting glycine with alanine or proline and vice versa as these residues often form part of the backbone structure.

Further, insertions or deletions may be made in the above sequences, e.g. in regions of the CDR loops that do not affect the direct antigen-binding interface. In addition, as is known in the art, rational design based on structural modeling may be used to predict acceptable mutations while retaining antigen-binding specificity.

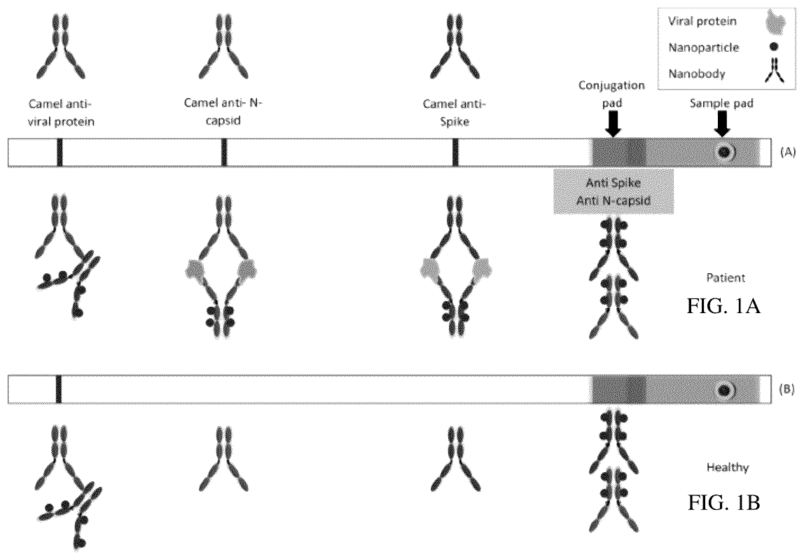

As shown in FIGS. 1A-B, after the target molecule binds to the detector HcAbs bound to nanoparticles, capillary action draws the sample across a porous membrane that is coated with capture HcAbs antibodies specific to the target molecule. This creates a visible or fluorescent signal that can be read by the naked eye or with a special instrument. An absorbent pad is attached at the end of the strip to wick the excess reagents and prevent backflow of the liquid. In some embodiments, the absorbent pad is made of cellulose fibers. The LFA test strip may be provided within a plastic or metal housing.

The sensitivity and specificity of an LFA depend on the quality and specificity of the capture and detector antibodies, as well as the efficiency of the nanoparticle conjugation. It is important to use high-quality antibodies and optimize the conjugation process to ensure accurate and reliable results. LFAs can be used for a variety of applications, including diagnosis of infectious agents or determination of immunity to specific diseases. They are particularly useful in resource-limited settings or for rapid point-of-care testing, as they do not require specialized equipment or trained technicians to operate. Results may be obtained rapidly, e.g. in 1-25 minutes.

Preferably, the HcAbs on the conjugate pad and the membrane specifically bind to the analyte. In one example, the test device is used in a sandwich assay format, in which HcAbs conjugated to detectable gold nanoparticles bind the analyte at the conjugate pad, and additional HcAbs immobilized on the membrane form a labeled HcAbs-analyte-HcAbs sandwich at the test line to generate a visible signal. The membrane may contain 1, 2, 3, or more test regions, each with different HcAbs that bind to a different antigen. The conjugate pad would contain HcAbs that bind to the same antigens as the HcAbs in the different test regions. For example, FIGS. 1 and 3 show two test regions which recognize either viral spike or viral capsid protein.

As used herein, the term “specifically binds” refers to the specificity of a binding reagent, e.g., an antibody, such that it preferentially binds to a defined analyte or target. Recognition by a binding reagent or an antibody of a particular analyte or target in the presence of other potential targets is one characteristic of such binding. In some embodiments, a binding reagent that specifically binds to an analyte avoids binding to other interfering moiety or moieties in the sample to be tested.

The control region is arranged at a point past the test regions and contains an anti-camelid IgG to ensure that the sample has migrated through the test regions.

Further embodiments provide an ELISA assay utilizing the nanobodies as described herein. For example, the nanobodies may be immobilized onto a selected surface, such as a well in a microplate (FIG. 6), e.g. a polystyrene microtiter plate. In some embodiments, the nanobodies are biotinylated and are immobilized on streptavidin-coated plates. A sample suspected of containing antigens, such as a clinical sample (e.g., a biological sample obtained from the subject), is added to the wells. After binding and/or washing to remove non-specifically bound immune complexes, the bound antigen may be detected. Detection is generally achieved by the addition of another nanobody that is linked to a detectable label (e.g. nanobodies conjugated to horseradish peroxidase (HRP)). This type of ELISA is a simple “sandwich ELISA.” Detection may also be achieved by the addition of a second antibody, followed by the addition of a third antibody that has binding affinity for the second antibody, with the third antibody being linked to a detectable label.

Various biological samples are suitable for testing in an assay as described herein including, but not limited to, whole blood, serum, plasma, urine, saliva, sweat, and other fluids. The sample may be applied directly to the device/well or pre-mixed with water, buffer, or other solution.

The sample may be obtained from a human or a non-human animal including, but not limited to dogs, horses, cats, rabbits, gerbils, hamsters, rodents, birds, aquatic mammals, cattle, pigs, camelids, and other zoological animals.

The present methods can be used for any suitable purpose. For example, present methods can be used for clinical diagnosis, prognosis, risk assessment and prediction, stratification and treatment monitoring and adjustment. In another example, present methods can be used for various research purposes, such as basic research, drug candidate screening, animal studies, and clinical trials. In still another example, present methods can be used in tests for standard setting, quality control, illegal drug screening, food safety, environmental safety, industrial safety, pollution, detection of biowarfare agents, screening for drugs or pharmaceuticals, and monitoring the quality of manufacturing using bioreactors looking for unwanted molecules, etc. The present tests devices and methods can be used in any suitable settings, such as tests in the labs, clinics, hospitals, physician's offices, homes, natural environments, battle fields and first responder environments, e.g., environments for fire, paramedic, police actions.

The present methods may further include a step of treating the subject who has been diagnosed with an assay as described herein. For example, if the LFA device or ELISA provides a positive result for SARS-COV-2 viral protein, then the subject is treated with an appropriate anti-viral therapy.

Embodiments of the disclosure also include methods of manufacturing an LFA device as described herein, e.g. via the steps set forth in the Example.

It is to be understood that this invention is not limited to any particular embodiment described herein and may vary. It is also to be understood that the terminology used herein is for the purpose of describing particular embodiments only, and is not intended to be limiting, since the scope of the present invention will be limited only by the appended claims.

Where a range of values is provided, it is understood that each intervening value between the upper and lower limit of that range (to a tenth of the unit of the lower limit) is included in the range and encompassed within the invention, unless the context or description clearly dictates otherwise. In addition, smaller ranges between any two values in the range are encompassed, unless the context or description clearly indicates otherwise.

Unless defined otherwise, all technical and scientific terms used herein have the same meaning as commonly understood by one of ordinary skill in the art to which this invention belongs. Representative illustrative methods and materials are herein described; methods and materials similar or equivalent to those described herein can also be used in the practice or testing of the present invention.

All publications and patents cited in this specification are herein incorporated by reference as if each individual publication or patent were specifically and individually indicated to be incorporated by reference, and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual dates of public availability and may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent”, or “except for [a particular feature or element]”, or “wherein [a particular feature or element] is not present (included, etc.) . . . ”.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order, which is logically possible.

### EXAMPLE 1

### Materials and Methods

### Materials

The process depicted in FIG. 4 outlines the overall scheme of the work being performed. SARS-COV-2 Spike S1 domain protein encoded DNA sequence and nucleocapsid protein encoding DNA sequence was synthesized by Synbiotech (New Jersey, USA), synthesized DNA was provided in pET28 vector cloned between XhoI and BamHI restriction sites. Freund's Incomplete Adjuvant was purchased from Sigma (cat number: F5506). Ni-Agarose was purchased from Cube Biotech (Germany, cat number: 75103).

Bovine serum albumin-—Sigma—Catalogue number: A3294-100; Gold nanoparticles 30 nm—Sigma—catalogue number: 753629—100ml; Sodium tetraborate decahydrate—Sigma—catalogue number: S9640; Whatman Standard 14 sample pads (Conjugate pad and sample pad) purchased from Cytiva (marlborouh, USA); Nitrocellulose membrane—FF120HP PLUS 210 catalogue number: 10547117 Cytiva; Absorbent pad—Axivia; Phosphate buffer saline tablets (PBS)—Sigma—Catalogue number: P4417 Sucrose—Sigma—Catalogue number: S0389.

### Methods

### Viral Protein Expression and Purification

pET21-Spike-S1 and pET21-N-cap vectors were transformed into B121 E. coli competent cells. Transformed cells selection, growth conditions and recombinant protein induction had been performed following the standard protocols (Maniatis Molecular Cloning A Laboratory Manual). Protein purification was performed using Cube Biotech standard native protein purification method for N-capsid and denatured protein purification for Spike S1 protein. Purified proteins were washed and concentrated using Amicon® Ultra-15 Centrifugal Filter Units (Membrane NMWL, 10 kDa). Washing and concentration step were against PBS solution for N-Cap and urea for Spike S1.

### Camel Immunization

A Bactrian camel (Camelus bactrianus) was immunized with 100 ug Spike S1 and another individual was immunized with 100 ug N-Cap protein by subcutaneous injection at five to six sites in the neck region. Institutional Animal Care and Use Committee guidelines were followed with animal subjects. Following primary injection using Freund complete adjuvant, four boosts were carried out at 3-week intervals using Freund incomplete adjuvant. Five milliliters of camel blood was collected before immunization and 3 days after each injection for serum titration for Spike S1 and N-Cap by indirect enzyme-linked immunosorbent assay (ELISA).

### Polyclonal HcAbs Purification

50 mg of Dynabeads® M-270 Epoxy (Thermo fisher, catalogue number: 14311D) were mixed with 50 ug Spike S1 and in another embodiment 50 mg of Dynabeads® M-270 Epoxy were mixed with 50 ug N-cap protein. Each reaction was mixed in a 1.5 mL micro centrifuge tube. The tubes were incubated at 37° C. overnight with continuous mixing in a rotary mixer at 50 rpm. The next day the magnetic beads were then washed and resuspended according to the manufacturer protocol. 50 mL of camel blood was collected in a heparin tube. The blood was centrifuged to isolate the serum and serum was collected for Spike S1 and N-cap immunized camels and mixed with the magnetic beads coupled with designated beads incubated at room temperature for 3 hours in a rotary mixer. The magnetic beads were then collected and washed with PBS buffer (1×) 4 times in magnetic field, then the purified antibodies were eluted using 0.1 M glycine, pH 2.0. Purified polyclonal antibodies were neutralized and tested using SDS-PAGE. The antibody solution was adjusted to 50 ug/mL and stored in 4° C. until used.

### Preparing Gold Nanoparticles HcAbs Conjugate

The gold nanoparticles (GNP) of 30 nm size were purchased in Sodium citrate buffer. GNP was washed 3 times using 2 mM Sodium tetraborate decahydrate buffer in Amicon Ultra-0.5 Centrifugal Filter 30 kDa MWCO Millipore. 400 mL GNP were collected in 2 mM Sodium tetraborate decahydrate, mixed with 60 pg of poly clonal HcAbs and incubated at 25° C. with constant mixing at 750 rpm for 1 hour. The GNP blocked with 2% BSA (10% cone.) was added to make a final BSA concentration of 1% and the mixture was incubated at 25° C. with constant mixing at 750 rpm for 1 hour. Following the blocking step, the mixture was washed 3 times using 2 mM Sodium tetraborate decahydrate buffer in Amicon Ultra-0.5 tubes then concentrated to 200 ul. This final solution is used for sensing the analyte dispensed at the conjugation pad.

### Spotting Capture HcAbs on a Membrane (Test Line)

1 pg of the purified HcAbs were used here as capture molecule. In this case, HcAbs specifically binds to Spike SI protein or N-Cap, and was mixed in distilled water with 1% sucrose and 1% trehalose buffer. Then, it was spotted on nitrocellulose membrane and dried at 37° C. for 2 hours. The dried membrane was blocked with 1% BSA for 15 minutes at room temperature and the membrane was dried again at 37° C. for 2 hours before use.

### Spotting the Control Line

The control line is composed here from rabbit anti-camel IgG on the nitrocellulose membrane. In this case, lpg of anti-camelid IgG antibodies mixed with 1% sucrose and trehalose buffer. This final mixture is used for membrane spotting and dried for 2 hours. The membrane was blocked as mentioned in the previous step.

### Sample Pad Preparation

The sample pad is soaked in 4% BSA for 10 minutes and dried at 37° C. until completely dried before being used in the assay.

### Preparation of Conjugation Pad

The gold nanoparticles conjugated to HcAbs are applied to a glass fiber conjugation pad, then allowed to dry at room temperature before use.

### Lateral Flow Assay

For the lateral flow assay, the sample pad is placed first, followed by the conjugation pad loaded with gold nanoparticles (GNP) conjugated with the HcAbs (human monoclonal antibodies). These two layers overlap and are followed by a nitrocellulose membrane with a test line on it. Finally, an adsorption pad is placed on top of the nitrocellulose membrane, overlapped with the preceding layers. To perform the assay, a sample is applied to the sample pad, which mixes with and binds to the GNP conjugate and flows towards the test line. If the sample contains the target analyte, it will be captured by the test line (FIGS. 3A-B), causing the color to develop into a red to purple shade. The assay also includes a control line, which is used to confirm the validity of the test by detecting the presence of a specific protein that produces a color development when bound to gold nanoparticle biosensors.

### EXAMPLE 2

### Sandwich ELISA and Affinity Test

The primary objective of this study was to develop and validate a sandwich enzyme-linked immunosorbent assay (ELISA) for the detection of SARS-COV-2 antigens, specifically the nucleocapsid protein (NCAP) and receptor-binding domain (RBD), using nanobodies. This approach aimed to explore the use of nanobodies as highly efficient, cost-effective, and specific detection agents compared to traditional antibodies. The goal was to enhance the capture efficiency of antigens by employing biotinylated nanobodies immobilized on streptavidin-coated plates, thereby improving assay sensitivity.

### Materials and Methods

### 1. Nanobody Design and ELISA Protocol

Capture Nanobodies: Nanobodies targeting the NCAP and RBD proteins were engineered with N-terminal histidine (His) tags for easier purification and handling. The secondary nanobodies were tagged with a hemagglutinin (HA) tag for detection.
ELISA Setup: A 96-well plate was coated with 400 ng/well of biotinylated anti-NCAP-1 or anti-RBD-1 nanobodies and incubated overnight at 4° C. After blocking with skimmed milk in phosphate-buffered saline with Tween-20 (PBS-T), SARS-COV-2 antigens (NCAP, RBD) or negative control (bovine serum albumin (BSA)) were added and incubated at 37° C. for 1 hour.
Detection Step: The antigens bound to the nanobodies were detected using nanobody-HRP fusion proteins, followed by the addition of 3,3′,5,5′-tetramethylbenzidine (TMB) and hydrogen peroxide (H₂O₂) to develop the signal, which was quantified by measuring absorbance at 450 nm.
Specificity Confirmation: Control wells without antigen showed no background signal, validating the specificity of the assay.

### 2. Detection Using Secondary Antibodies

The HA-tagged secondary nanobodies bound to the captured antigen-nanobody complexes. These complexes were detected using a commercially available anti-HA-HRP antibody (Roche, 1:5000), which specifically recognized the HA tag on the nanobodies.
   3. Optimal Nanobodies Based on ducking-Based Affinity for SARS-COV-2 Antigens: Sequences and Paratopes of NCAP and RBD Nanobodies

### COVID Nanobodies Sequences

a. N Capsid nanobodies
>NCAP-1
(SEQ ID NO: 5)
QVQLVESGGGSVQAGGSLRLSCIASGTFPFRFNAMAWFRQAPGNQRELVA
GIFTTYATNYAESVKGRFTISRDNEKNTIYLQMNSLKPEDTGVYYCNAAY
VSTSIWRPLYEEWGQGTQVTVSS
Paratope
(SEQ ID NO: 6)
GLPYGANKDGIIWVATEGALNTPKDHIGTRNPANNAAIVLQLPQGTTLPK
GFYAEGSRGGSQASSRSSSRSRNSSRNSTPGSSRGTSPARMAGNGGDAAL
ALLLLDRLNQLE
>NCAP-2
(SEQ ID NO: 7)
QVQLVESGGALMQAGGSLRLSCAVSGIRLGYYTIAWFRQAPGKEREGISC
ISRSEGSTYYADSVKGRFTISTENAENTVYLQMNSLKPDDTAVYYCAVDF
VPASHCAVANTKGYDYWGQGTQVTVSS
Paratope
(SEQ ID NO: 8)
MSDNGPQNQRNAPRITFGGPSDSTGSNQNGERSGARSKQRRPQGLPNNTA
SWFTALTQHGKEDLKFPRGQGVPINTNSSPDDQIGYYRRATRRIRGGDGK
b. RBD nanobodies
>RBD-1
(SEQ ID NO: 9)
QVQLVESGGGSVQAGGSLRLSCAASGSISSITYLGWFRQAPGKEREGVAA
LITVQAHTYYADSVKGRFTISRDNAKNTVYLQMNSLKPEDTALYYCAAAA
FGYAWPLRQDDYWYWGQGTQVTVSS
Paratope
(SEQ ID NO: 10)
YNSASFSTFKCYGVSPTKLNDLCFTNVYADSFVIRGDEVRQIAPGQTGKI
ADYNYKLPDDFTGCVIAWNSNNL
>RBD-2
(SEQ ID NO: 11)
QVQLVESGGGSVQAGGSLRLSCIGSARTFHSYVMAWFRQAPGKEREFVAA
ISWSSTPTYYGESVKGRFTISRDNAKNTVYLQMNSLKPEDTAVYFCAADR
ADSYYHTRPTDYEFWGQGTQVTVSS
Paratope
(SEQ ID NO: 12)
QAGSTPCNGVEGENCYFPLQSYGFQPTNGVGYQ

### Results

The protein sequence information presented above details the sequences of nanobodies targeting two key antigens of the SARS-COV-2 virus, the nucleocapsid protein (NCAP) and the receptor-binding domain (RBD) of the spike protein. These nanobodies were selected based on their antigen-binding affinity predicted through computational (docking) analysis to the respective antigens, indicating their use for high specificity and binding efficacy in diagnostic applications.

### Nanobodies Targeting NCAP (Nucleocapsid Protein)

### >NCAP-1

Sequence: The sequence of NCAP-1 is provided in the first part, starting with the variable region of the heavy chain (V_H) that defines the antibody's specificity. It includes key amino acid residues for antigen recognition.
Paratope: The paratope is the specific region of the antibody that interacts directly with the antigen. The sequence for the paratope of NCAP-1 is shown, detailing the amino acid residues that form the binding site for NCAP. This region plays a critical role in recognizing and binding to the SARS-COV-2 nucleocapsid protein.

### >NCAP-2

Sequence: Similarly, the sequence for NCAP-2 is shown, with some variations in the amino acid composition compared to NCAP-1, which may affect its binding characteristics or specificity to the NCAP antigen.
Paratope: The paratope sequence for NCAP-2 is provided, illustrating the specific set of residues involved in antigen binding. These sequences highlight how the nanobody's structure is tailored to recognize the NCAP antigen effectively.

### Nanobodies Targeting RBD (Receptor-Binding Domain)

### >RBD-1

Sequence: The sequence for RBD-1 begins with the variable domain for antigen recognition. It is designed to specifically interact with the receptor-binding domain (RBD) of the SARS-COV-2 spike protein, an important site for viral entry into host cells.
Paratope: The paratope sequence of RBD-1 is provided, showing the amino acid residues that specifically interact with the RBD antigen. This information is critical for understanding how RBD-1 binds to the spike protein and may inform its application in diagnostics or therapeutics.

### >RBD-2

Sequence: The RBD-2 nanobody sequence is similar to RBD-1 but with slight variations that may confer different binding properties, potentially enhancing its affinity or specificity for the RBD antigen.
Paratope: The paratope sequence for RBD-2 is also provided, detailing the residues responsible for binding to the RBD antigen. These residues are crucial in determining the effectiveness of RBD-2 as a potential detection agent for SARS-COV-2.
   The provided information focuses on the sequences of the nanobodies (both NCAP and RBD-specific) and their paratopes, offering a detailed view of their utility in diagnostics. The sequences are designed for high affinity and specificity to the SARS-COV-2 antigens. Bioinformatics-based selection has been used to identify these nanobodies as candidates for efficient antigen detection.

### Validation of Nanobody Pairs for Sensitive Detection of SARS-COV-2 NCAP and RBD Proteins in ELISA Assays.

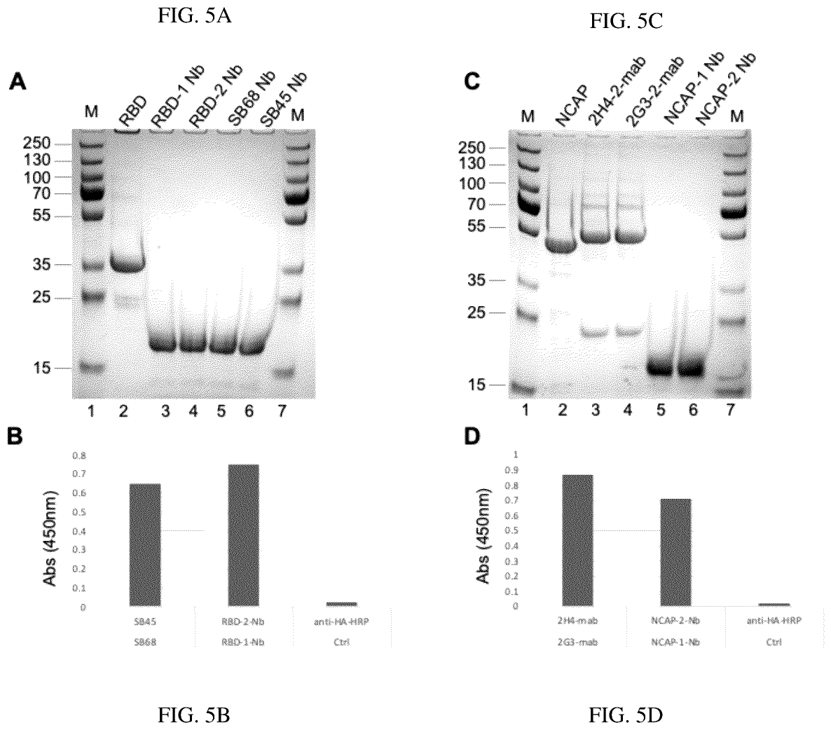

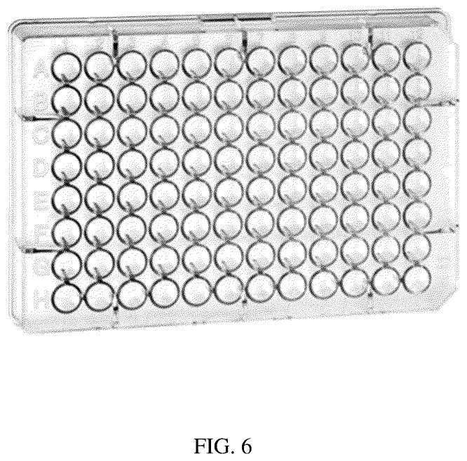

SDS-PAGE Analysis: The purity and integrity of the recombinant NCAP and RBD proteins were confirmed via SDS-PAGE, validating their effective use in antigen capture and detection steps (FIGS. 5A and 5C).
Signal Intensity and Specificity: The anti-NCAP-1/NCAP-2 nanobody pair demonstrated strong binding to NCAP, with signal intensity comparable to that of a monoclonal antibody pair (anti-NCAP 2H4/2G3), as shown in FIG. 5B. Similarly, the anti-RBD-1/RBD-2 nanobody pair exhibited higher signal intensity than the monoclonal antibody pair SB68/SB45 (FIG. 5D), which was previously published by Chen et al. (2021).
Antigen Capture and Detection: The biotin-streptavidin interaction enhanced the capture efficiency of the nanobodies, allowing for effective antigen detection without significant background signals in negative control wells.

### Conclusion

The development of nanobody-based diagnostic assays for SARS-COV-2 demonstrates the use of these agents for pathogen detection. The sandwich ELISA described here, utilizing biotinylated nanobodies immobilized on streptavidin-coated plates, offers significant advantages over traditional antibody-based assays in terms of sensitivity, specificity, and cost-effectiveness. The findings underscore the value of nanobodies in diagnostic applications, paving the way for their use in both diagnostic and therapeutic settings.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

### Acknowledgement

The inventors extend their appreciation to the Deputyship for Research & Innovation, Ministry of Education in Saudi Arabia for funding this research work through the project number (IFPNC-011-130-2020) and King Abdulaziz University, DSR Jeddah, Saudi Arabia.

What is claimed is:

1. A lateral flow assay (LFA) device for pathogen detection, comprising
a sample pad at a first end of the device for receiving a sample;
a conjugate pad containing gold nanoparticles conjugated to heavy chain antibodies (HcAbs), wherein the conjugate pad partially overlaps the sample pad;
a porous membrane containing a test region and a control region; and
an absorbent pad at a second end of the device,
wherein the porous membrane partially overlaps the conjugate pad and the absorbent pad,
wherein each of the sample pad, conjugate pad, porous membrane, and absorbent pad are at least partially mounted on a solid support, and
wherein the HcAbs comprise three complementarity determining regions (CDRs) having a sequence at least 90% identical to CDR sequences as set forth in SEQ ID NO: 5, SEQ ID NO: 7, SEQ ID NO: 9, or SEQ ID NO: 11.

2. The LFA device of claim 1, wherein the HcAbs comprise three CDRs consisting of CDR sequences as set forth in SEQ ID NO: 5, SEQ ID NO: 7, SEQ ID NO: 9, or SEQ ID NO: 11.

3. The LFA device of claim 1, wherein the control region comprises immobilized anti-camel IgG.

4. The LFA device of claim 1, wherein the test region comprises immobilized capture HcAbs and wherein the immobilized capture HcAbs comprise three CDRs consisting of CDR sequences as set forth in SEQ ID NO: 5, SEQ ID NO: 7, SEQ ID NO: 9, or SEQ ID NO: 11.

5. The LFA device of claim 1, wherein the porous membrane is a nitrocellulose membrane.

6. A method of detecting a pathogen in a biological sample, comprising contacting the biological sample with an LFA device of claim 1.

7. A heavy chain antibody, comprising a paratope having a sequence at least 90% identical to CDR sequences as set forth in SEQ ID NO: 5, SEQ ID NO: 7, SEQ ID NO: 9, or SEQ ID NO: 11.

8. The heavy chain antibody of claim 7, wherein the heavy chain antibody comprises three CDRs consisting of CDR sequences as set forth in SEQ ID NO: 5, SEQ ID NO: 7, SEQ ID NO: 9, or SEQ ID NO: 11.

9. The heavy chain antibody of claim 7, wherein the heavy chain antibody is immobilized in a well of a microplate.

10. The heavy chain antibody of claim 9, wherein the heavy chain antibody is biotinylated and is bound to streptavidin coating the well.

11. A method of detecting a pathogen in a biological sample, comprising contacting the biological sample with the heavy chain antibody of claim 9.
